(19) United States
(12) Patent Application Publication
(10) Pub. No.: US 2012/0021921 A1
(43) Pub. Date: Jan. 26, 2012

(54) PROCESS AND METHOD FOR MONITORING GASTROINTESTINAL MICROBIOTA

(76) Inventors: David L. Scott, Conyers, GA (US); James Alexander Bralley, III, Johns Creek, GA (US); Joseph Marshall George, Sugar Hill, GA (US)

(21) Appl. No.: 12/416,905

(22) Filed: Apr. 1, 2009

Related U.S. Application Data
(60) Provisional application No. 61/041,581, filed on Apr. 1, 2008.
(60) Provisional application No. 61/041,584, filed on Apr. 1, 2008.

(51) Int. Cl. C12Q 1/68; C40B 30/00

(52) U.S. Cl. 506/7; 435/6.12; 435/6.11; 435/6.15

(57) ABSTRACT

Disclosed are methods for monitoring the gastrointestinal tract of the human gastrointestinal system. The method includes: 1) grouping microbes into specific operational taxonomic units (OTU); 2) using oligonucleotide probes and PCR primers to detect and quantify specific microbes (bacteria, fungi/yeast, protozoans and parasitic worms) in human fecal material. The inventions also discloses a kit that includes: a DNA isolation step; 2) accumulation of specific operational taxonomic units (OTU); 3) identification and quantifying of sequences internal to the OTU; 4) reporting changes the indigenous population of the human gastrointestinal system.

### CROSS REFERENCE TO A PROVISIONAL APPLICATION

This application claims the benefit of Provisional Application Ser. No. 61/041,581, filed on Apr. 1, 2008, and Provisional Application Ser. No. 61/041,584, also filed on Apr. 1, 2008, and the entirety of each is hereby incorporated herein by reference.

### SEQUENCE LISTING

This application includes a Sequence Listing presented herewith. Filed herewith is electronic file “GI Sequences_ST25.txt” created Apr. 1, 2009, with a size of 48 KB, the entirety of which is hereby incorporated herein by reference.

### BACKGROUND OF THE INVENTION

1. Field of the Invention

The present invention relates to the use specific oligonucleotide probes and PCR primers in molecular-based methods to detect and quantify microbes indigenous and pathogenic to the human gastrointestinal tract.

2. Background of the Invention

The following literature is of use in the subject matter of the present invention and is incorporated herein by reference:
1. Mackie R I, Sghir A, Gaskins H R. Developmental microbial ecology of the neonatal gastrointestinal tract. Am J Clin Nutr. May 1999; 69(5):1035S-1045S.
2. Hawrelak J A, Myers S P. The causes of intestinal dysbiosis: a review. Altern Med. Rev. June 2004; 9(2):180-197.
3. Galland L, Barrie S. Intestinal dysbiosis and the causes of diseases. J. Advancement Med. 1993; 6:67-82.
4. Savage D C. Microbial ecology of the gastrointestinal tract. Annu Rev Microbiol. 1977; 31:107-133.
5. Berg R D. The indigenous gastrointestinal microflora. Trends Microbiol. November 1996; 4(11):430-435.
6. Finegold S, Sutter V, Mathisen G. Normal indigenous intestinal flora. New York: Academic Press; 1983.
7. Leff et al., 1995, Appl. Environ. Microbiol., 61:1634-1636.
8. Xiao et al, 1999, Appl Environ. Microbiol., 65:3386-3391.

The population of the microbiota of the human gastrointestinal (“GI”) tract is widely diverse and complex with a high population density. All major groups of organisms are represented. While predominately bacteria, a variety of protozoa are also present. In the colon there are over 10¹¹ bacterial cells per gram and over 400 different species. These bacterial cells outnumber host cells by at least a factor of 10. This microbial population has important influences on host physiological, nutritional and immunological processes. In particular, they protect against pathogenic bacteria and drive the development of the immune system during neonatal life. Further metabolic activities of the GI microbiota that beneficially affect the host include continued degradation of food components, vitamin production, and production of short chain fatty acids that feed the colonic mucosa. It is clear that factors such as diet, sickness, stress, or medication can result in loss of well-being of the host, and it is assumed that some of these symptoms are due to perturbation of what is termed the normal balance of the gut microbiota. Knowledge of the structure and function of the standard microbiota, and its response to diet, genetic background and lifetime of the host must be taken into account when designing probiotic-based functional foods. Moreover, this biomass should more rightly be considered a rapidly adapting, renewable organ with considerable metabolic activity and significant influence on human health. Consequently there is renewed and growing interest in identifying the types and activities of these gut microbes.¹

The normal, healthy balance in microbiota provides colonization resistance to pathogens. Since anaerobes comprise over 95% of these organisms, their analysis is of prime importance. Gut microbes might also stimulate immune responses to prevent conditions such as intestinal dysbiosis. Intestinal dysbiosis may be defined as a state of disordered microbial ecology that causes disease. Specifically, the concept of dysbiosis rests on the assumption that patterns of intestinal flora, specifically overgrowth of some microorganisms found commonly in intestinal flora, have an impact on human health. Symptoms and conditions thought to be caused or complicated by dysbiosis include inflammatory bowel diseases, inflammatory or autoimmune disorders, food allergy, atopic eczema, unexplained fatigue, arthritis, mental/emotional disorders in both children and adults, malnutrition and breast and colon cancer.^{2,3}

Most studies of microbiota in the GI tract have used fecal samples. These do not necessarily represent the populations along the entire GI tract from stomach to rectum. Conditions and species can alter greatly along this tract and generally run from lower to higher population densities. The stomach and proximal small intestine with highly acid conditions and rapid flow contain 10³ to 10⁵ bacteria per gram or ml of content. These are predominated by acid tolerant lactobacilli and streptococci bacteria. The distal small intestine to the ileocecal valve usually ranges to 10⁸ bacteria per gram or ml of content. The large intestine generates the highest growth due to longer residence time and ranges from 10¹⁰ to 10¹¹ bacteria per gram or ml of content. This region generates a low redox potential and high amount of short chain fatty acids.

Not only does the microbiota content change throughout the length of the GI tract but there are also different microenvironments where these organisms can grow. At least four microhabitats exist: the intestinal lumen, the unstirred mucus layer that covers the epithelium, the deeper mucus layer in the crypts between villi, and the surface mucosa of the epithelial cells.^{4,5} Given this diverse ecological community the question arises as to how to sample the various environments to identify populations of microbes and ultimately understand the host-microbe interactions. This problem is an extremely difficult one since any intervention to obtain a sample potentially disrupts the population. Fecal sampling has been used for years in microbiota assessment. But it should be understood that this sample primarily most appropriately represents organisms growing in the colon. In addition, >98% of fecal bacteria will not grow in oxygen.⁴ Therefore, standard culture techniques miss the majority of organisms present.

Conventional bacteriological methods like microscopy, culture, and identification are used for the analysis and/or quantification of the intestinal microbiota.⁶ Limitations of conventional methods are their low sensitivities,⁷ their inability to detect noncultivatable bacteria and unknown species, their time-consuming aspects, and their low levels of reproducibility due to the multitude of species to be identified and quantified. In addition, the large differences in growth rates and growth requirements of the different species present in the human gut indicate that quantification by culture is bound to be inaccurate. The application of molecular techniques for detection and identification of microbes has provided a major breakthrough in the analysis of microbial ecosystems and their function.⁷

To overcome the problems of culture, a number of molecular-based methods have been employed to characterize the microbiota of the human gastrointestinal tract. Although identification and characterization of genomic sequence data for individual microbes may provide for the identification of specific microbes, such targeted testing fails to provide a comprehensive, economically feasible system for monitoring the ecosystem of the gastrointestinal tract. The accuracy of a molecular diagnostic test for a microbe may be compromised where the pathogenic agent is endemic, or possesses substantial genetic similarity to non-pathogenic organisms.^{7,8}

Detailed information of the microbial community composition in natural systems can be gained from the phylogenetic analysis of 16S rDNA sequences obtained directly from samples by PCR amplification, cloning and sequencing. However, the results showed that the microbial community is complex, and that the bacterial diversity cannot be comprehended by culturing.⁸

Considering the aforementioned, there is an obvious need in the art for process and methods that enable real-time monitoring of the balance of indigenous microorganisms of the human gastrointestinal tract. The monitoring system should also allow for detection of known, as well as unknown, pathogenic microbes that may have a negative impact on human health.

### SUMMARY OF THE INVENTION

In one aspect the present invention provides a method for monitoring the microbiota of the human gastrointestinal tract. The method includes the steps of identifying universal PCR primers to group microbial operational taxonomic units, and then applying the universal PCR primers to a sample of the gastrointestinal tract to produce PCR products between 500 bp-1500 bp in size. In another aspect, the universal PCR primers are specific to bacteria operational taxonomic units and include the sequence of any one of SEQ ID NO:1-SEQ ID NO:2 and SEQ ID NO. 54-SEQ ID NO. 55. In another aspect, the universal PCR primers are specific to fungi and yeast operational taxonomic units and include the sequence of any one of SEQ ID NO:82-SEQ ID NO:83 and SEQ ID NO:92-SEQ ID NO:93. In yet another aspect, the universal PCR primers are specific to parasitic protozoans and worms operational taxonomic units and include the sequence of any one of SEQ ID NO:92-SEQ ID NO:93.

In another aspect, the universal PCR primers obtain qualitative or quantitative data and report for specific microbial DNA sequences by analyzing DNA sequences of specific microbial operational taxonomic units using molecular-based methods. The molecular based methods may include DNA hybridization, DNA arrays, DNA sequencing, PCR Arrays and multiplex PCR. The oligonucleotides probes may include sequences of any one of SEQ ID NO:1-SEQ ID NO:309 for the differentiation of microbes localized to the internal sequences of a specific operational taxonomic unit.

In yet another aspect, the invention provides a process for monitoring microorganisms that are indigenous and/or pathogenic to an ecosystem. The process including providing a method for simultaneous collection and inactivation of microbial growth in fecal material, providing a method for extracting DNA from fecal material that is amendable to sensitive nucleic acid analysis, and providing a method for concentrating target microbial nucleic acids. The process then provides for the specific identification and quantification of nucleic acid sequences specific to a microorganism at the genus or species level. The ecosystem of interest may include the human gastrointestinal tract. The fecal material may be collected in medium containing 0.1%-50% formalin and the target nucleic acid may be DNA.

In yet another aspect, the present invention provides a method for detecting a microbial species in a sample. The method includes the steps of lysing cells in said sample to release genomic DNA. Contacting genomic DNA from the previous step with a primer pair comprising sequences of any one of SEQ ID NO:1-SEQ ID NO:309 for the differentiation of microbes localized to the internal sequences of a specific operational taxonomic unit. Amplifying the microbial DNA to produce an amplification product. And detecting said amplification product wherein the presence of said product is indicative of the presence of a microbial species in said sample and the absence of said product is indicative of the absence of a microbial species in said sample. The method may also include quantitating the level of a microbial species in the sample. The method includes the steps of quantitating the level of said amplification product by comparison with at least one reference standard, wherein the level of said amplification product is indicative of the level of said microbial species.

These and other aspects of the invention will become apparent from the following description of the preferred embodiments taken in conjunction with the tables and figures. As would be obvious to one skilled in the art, many variations and modifications of the invention may be effected without departing from the spirit and scope of the novel concepts of the disclosure.

### DETAILED DESCRIPTION OF PREFERRED EMBODIMENTS

Because of the demand for screening test that are rapid for pathogen and antibiotic resistance identification, molecular diagnostics are playing an increasingly important role in diagnosing and preventing infections and improving overall hospital operations. As physicians, pharmacists and even hospitals administrators demand rapid microbiology results, many laboratories are focusing on being part of cross-functional implementation teams that not only assure the new tests are implemented efficiently, but that the results affect real change for patient management, hospital operations and laboratory efficacy. The present invention provides a process for monitoring the microbial populations of the human gastrointestinal tract. To improve our understanding of the intestinal ecosystem the present invention takes a ribosomal RNA-approach targeting the small and large-subunit rRNA's with various molecular methods, each having its advantages. The present invention may be embodied in a variety of ways.

According to a first embodiment of the invention, there is provided a consortium of microorganisms indigenous and/or pathogenic to the human gastrointestinal tract. This embodiment comprises a method to prepare a DNA sample from fecal material preserved in formalin, the method comprises grouping the DNA sequences into operational taxonomic units (OTUs) using universal PCR primers. The primers used to detect microbial operational taxonomic units are presented in the Sequence Listing below.

The combination of the non-specific fragmenting genomic DNA by formalin and the DNA isolation method used the aforementioned universal PCR primers disclosed in this invention are design to amplify target sequences that are between 500-1200 base pairs. Moreover these primers flank regions of high sequence heterogeneity that allows the differentiation of microbial organism at the genus/species level.

The method may include identifying at least one nucleic acid sequence that is specific to a single OTU isolated nucleic acid having a sequence derived from a single predetermined microbial operational taxonomic unit. The microbial operational taxonomic unit PCR primers are disclosed in this invention for bacteria, fungi/yeast, protozoan's, and parasitic worms.

According to the first embodiment of the invention, there is provided a primer pair for PCR amplification of bacteria DNA, said primer pair comprising: (a) a first oligonucleotide of at least 18 nucleotides having a sequence selected from one strand of a bacterial 16S rDNA gene; and (b) a second oligonucleotide of at least 18 nucleotides having a sequence selected from the other strand of said 16S rDNA gene downstream of said first oligonucleotide sequence; wherein at least one of said first and second oligonucleotides is selected from: (i) any one of SEQ ID NO: 1 to SEQ ID NO: 2; or (ii) a DNA sequence having at least 92% identity with any one SEQ ID NO: 1 to SEQ ID NO: 2.

According to another embodiment of the present invention, there is provided a primer pair for PCR amplification of Bacteria DNA, said primer pair comprising: (a) a first oligonucleotide of at least 18 nucleotides having a sequence selected from one strand of a bacterial 23S rDNA gene; and (b) a second oligonucleotide of at least 18 nucleotides having a sequence selected from the other strand of said 23S rDNA gene downstream of said first oligonucleotide sequence; wherein at least one of said first and second oligonucleotides is selected from: (i) any one of SEQ ID NO: 54 to SEQ ID NO: 55; or (ii) a DNA sequence having at least 92% identity with any one SEQ ID NO: 54 to SEQ ID NO: 55.

According to another embodiment of the present invention, there is provided a primer pair for PCR amplification of fungi/yeast DNA, said primer pair comprising: (a) a first oligonucleotide of at least 18 nucleotides having a sequence selected from one strand of a fungus or yeast 18S rDNA gene; and (b) a second oligonucleotide of at least 12 nucleotides having a sequence selected from the other strand of said 18S rDNA gene downstream of said first oligonucleotide sequence; wherein at least one of said first and second oligonucleotides is selected from: (i) any one of SEQ ID NO: 82 to SEQ ID NO: 83; or (ii) a DNA sequence having at least 92% identity with any one SEQ ID NO: 82 to SEQ ID NO: 83.

According to another embodiment of the present invention, there is provided a primer pair for PCR amplification of fungi, protozoan and parasitic worm DNA, said primer pair comprising: (a) a first oligonucleotide of at least 18 nucleotides having a sequence selected from one strand of a protozoan/worm 18S rDNA gene; and (b) a second oligonucleotide of at least 12 nucleotides having a sequence selected from the other strand of said 18S rDNA gene downstream of said first oligonucleotide sequence; wherein at least one of said first and second oligonucleotides is selected from: (i) any one of SEQ ID NO: 92 to SEQ ID NO: 93; or (ii) a DNA sequence having at least 92% identity with any one SEQ ID NO: 92 to SEQ ID NO: 93.

According to yet another embodiment, the present invention may provide a method for monitoring the microbiota of the human gastrointestinal tract whereby quantitative and qualitative data can be provided by using quantifiable labels to label the universal PCR primers that represent individual or all of the microbial operational taxonomic units disclosed in this invention. Furthermore, these labeled operation taxonomic units in conjunction with a plurality (SEQ ID NO:1 thru SEQ ID NO:309) of available oligonucleotide probes (40 bp-100 bp) that are localize internally to the disclosed universal sequences may be used in DNA hybridization or array based methods to provide information on the abundance of specific organisms of interest, such as key bioindicators, pathogens, or microbial contaminants in a gastrointestinal tract system.

In yet another embodiment of the present invention, there is provided a kit for monitoring the microbiota of the human gastrointestinal tract comprising: at least one primer according to an embodiment of the invention; or at least one primer pair according to another embodiment of the invention; or at least one probe according to yet another embodiment of the invention.

### Examples

The primers used to detect microbial operational taxonomic units are presented in the Sequence Listing.

### Universal Bacteria PCR

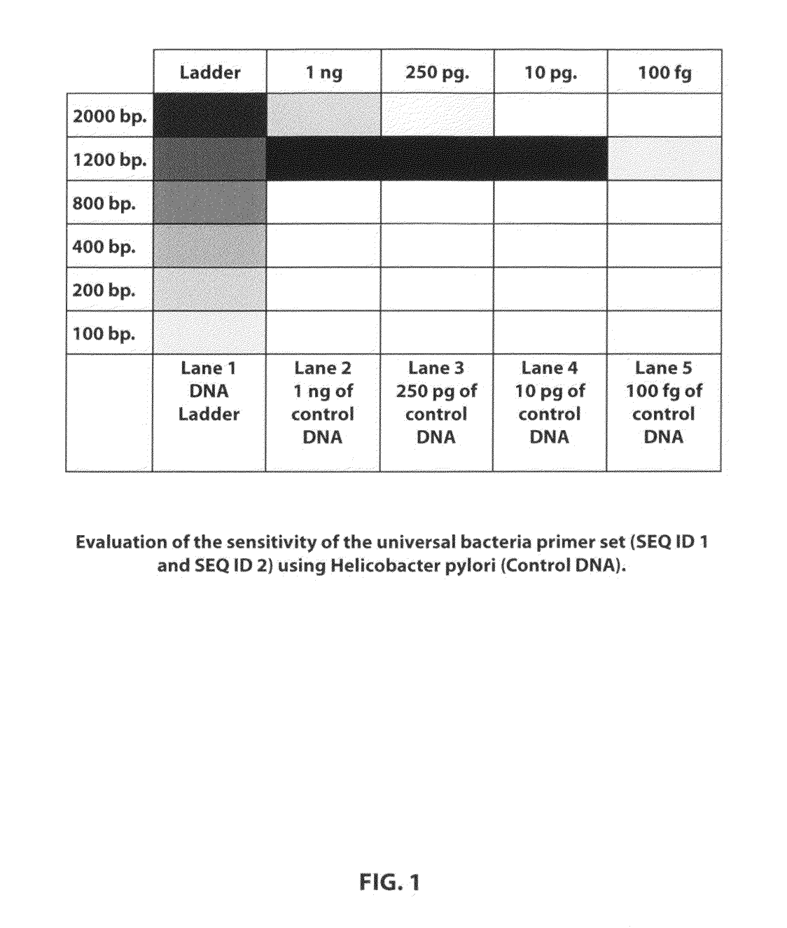

The melting temperature calculated for entbac1 (SEQ ID NO:1) was 60 degree C. and a fragment size of approximately 1052 nucleotides was calculated in a PCR with primer (SEQ ID NO:2). The entbac2 (SEQ ID NO:2) sequence corresponds to the sequence at positions 440 to 457 of the E. coli 16S rDNA gene. The PCRs were carried out according to methods detailed in “Molecular Cloning: a Laboratory Manual” Sambrook et al. 2nd ed. Cold Spring Harbor Laboratory Press, Cold Spring Harbor, N.Y. (1989) which is incorporated herein by reference, at an annealing temperature of 55 degrees C. The results of electrophoretic analysis of PCRs on an agarose gel are presented in FIG. 1. Details of the material analysed in each lane of the gel are given in FIG. 1. The results depicted in FIG. 1 are tabulated below in Table 1.

TABLE 1
Evaluation of the sensitivity of the universal bacteria primer set
(SEQ ID 1 and SEQ ID 2) using Helicobacter pylori (Control DNA).

| Lane | Scoring |
|---|---|
| Lane 1 (1 ng) | +++ |
| Lane 2 (250 pg) | ++ |
| Lane 3 (10 pg) | + |
| Lane 4 (100 fg) | +/− |

The scorings for the agarose gel electrophoresis analysis is by quantitating the intensity of the PCR products in the stained gel using the naked eye. A definition of the scoring follows: − = no band; +/− = very faint band; + through ++++ = increasing intensity of the PCR products.

Amplification of Universal Bacteria DNA from Different Transport Medium

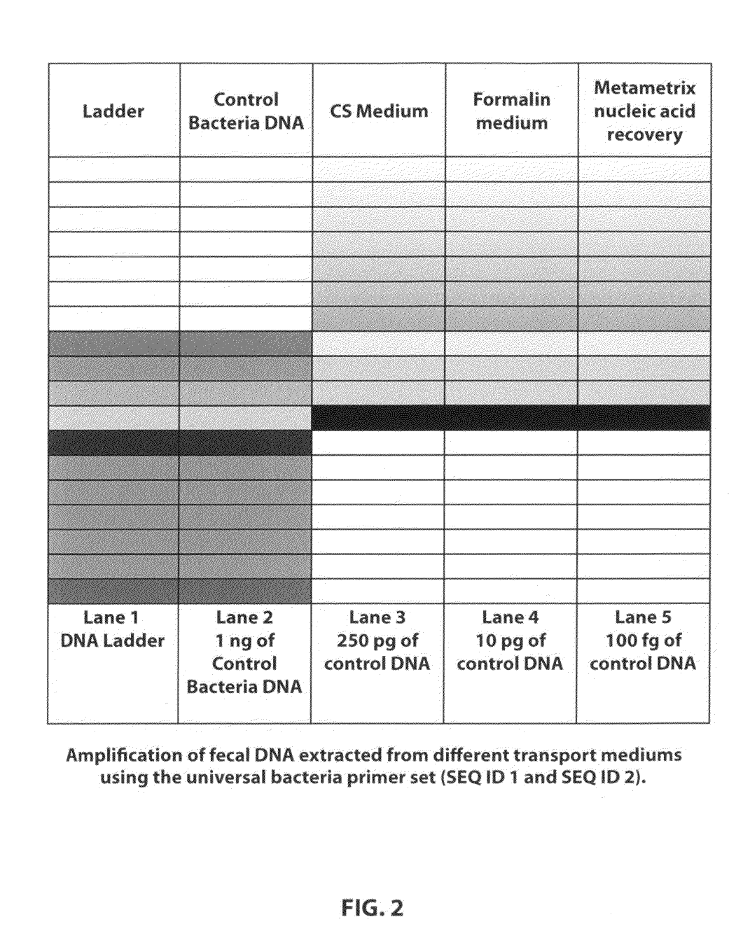

The bacterial universal primer pairs were used to amplify DNA extracted from 3 different transport mediums and the results are presented in FIG. 2. The PCRs were carried out according to methods detailed in Sambrook et al. (1989) at an annealing temperature of 55 degrees C. The results of electrophoretic analysis of PCRs on an agarose gel are presented in FIG. 2. Details of the material analysed in each lane of the gel are given in FIG. 2. The results depicted in FIG. 2 are tabulated below in Table 2.

TABLE 2
Amplification of fecal DNA extracted from different
transport mediums using the universal bacteria
primer set (SEQ ID 1 and SEQ ID 2).

| Lane | Scoring |
|---|---|
| Lane 1 (CS medium) | +++ |
| Lane 2 (Formalin medium) | +++ |
| Lane 3 (Metametrix Nucleic Acid | +++ |
| Recovery Solution) | |

The scorings for the agarose gel electrophoresis analysis is by quantitating the intensity of the PCR products in the stained gel using the naked eye. A definition of the scoring follows: − = no band; +/− = very faint band; + through ++++ = increasing intensity of the PCR products.

### Evaluation of the Specificity of the Universal Bacteria DNA

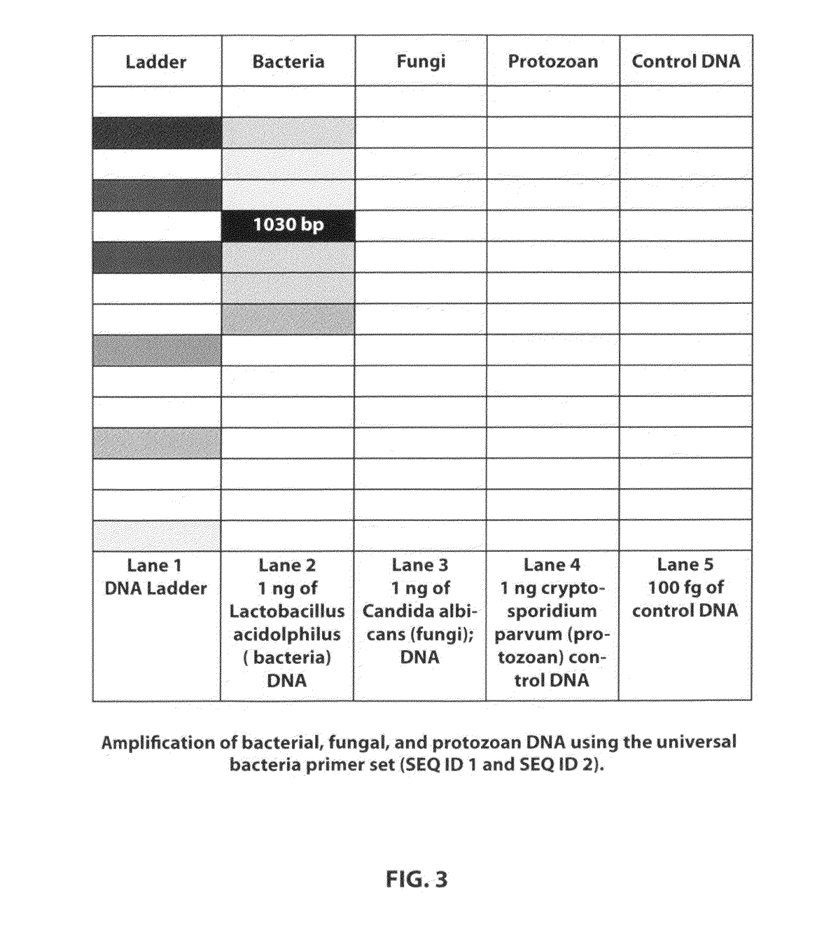

The bacterial universal primer pairs were used to amplify DNA from bacteria (Lactobacillus), protozoan (cryptosporidium parvum), and fungal (Candidia albicans) to evaluated the specificity of the primer set. The PCR's were carried out according to methods detailed in Sambrook et al. (1989) at an annealing temperature of 55 degrees C. The results of this assay are presented in FIG. 3. The results of electrophoretic analysis of PCRs on an agarose gel are presented in FIG. 3. Details of the material analysed in each lane of the gel are given in FIG. 3. The results depicted in FIG. 3 are tabulated below in Table 3.

TABLE 3
Amplification of bacterial, fungal, and protozoan DNA using
the universal bacteria primer set (SEQ ID 1 and SEQ ID 2).

| Lane | Scoring |
|---|---|
| Lane 1 (Bacteria DNA) | +++ |
| Lane 2 (Fungi DNA) | − |
| Lane 3 (Protozoan DNA) | − |

The scorings for the agarose gel electrophoresis analysis is by quantitating the intensity of the PCR products in the stained gel using the naked eye. A definition of the scoring follows: − = no band; +/− = very faint band; + through ++++ = increasing intensity of the PCR products.

### Evaluation of the Specificity of Oligonucleotide Probes in a PCR Assay

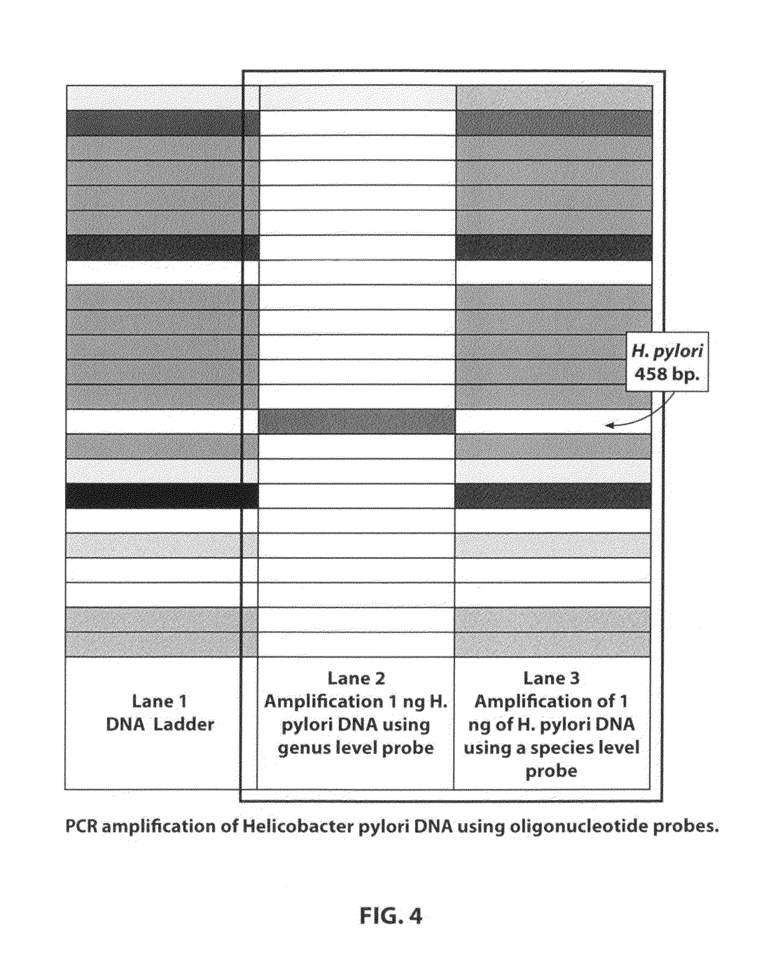

The primer for the specific detection of Helicobacter pylori (SEQ ID NO: 283) was used in a diagnostic PCR. The primer was designed originally for the hybridization experiments. The specificity of this primer can be appreciated from the sequence alignment presented in FIG. 4 which is an alignment of 16S rDNA sequences of bacterial species localized to the human GI tract against (SEQ ID NO: 283). A melting temperature of 60 degrees C. was calculated for the primer (SEQ ID NO: 50) and a fragment size of approximately 356 nucleotides in a PCR with the forward primer (SEQ ID NO:282) used for the specific detection of H. pylori as experimentally determined. The PCRs were carried out according to methods detailed in Sambrook et al. (1989) at an annealing temperature of 50 degrees C. The results of electrophoretic analysis of PCRs on an agarose gel are presented in FIG. 4. Details of the material analysed in each lane of the gel are given in FIG. 4. The results depicted in FIG. 4 are tabulated below in Table 4.

TABLE 4
PCR amplification of Helicobacter pylori
DNA using oligonucleotide probes.

| Lane | Scoring |
|---|---|
| Lane 1 (helicobacter genus probe) | +++ |
| Lane 2 (H. pylori specific probe) | +++ |

The scorings for the agarose gel electrophoresis analysis is by quantitating the intensity of the PCR products in the stained gel using the naked eye. A definition of the scoring follows: − = no band; +/− = very faint band; + through ++++ = increasing intensity of the PCR products.

Amplification of Universal Bacteria DNA Extracted from Human Fecal Material.

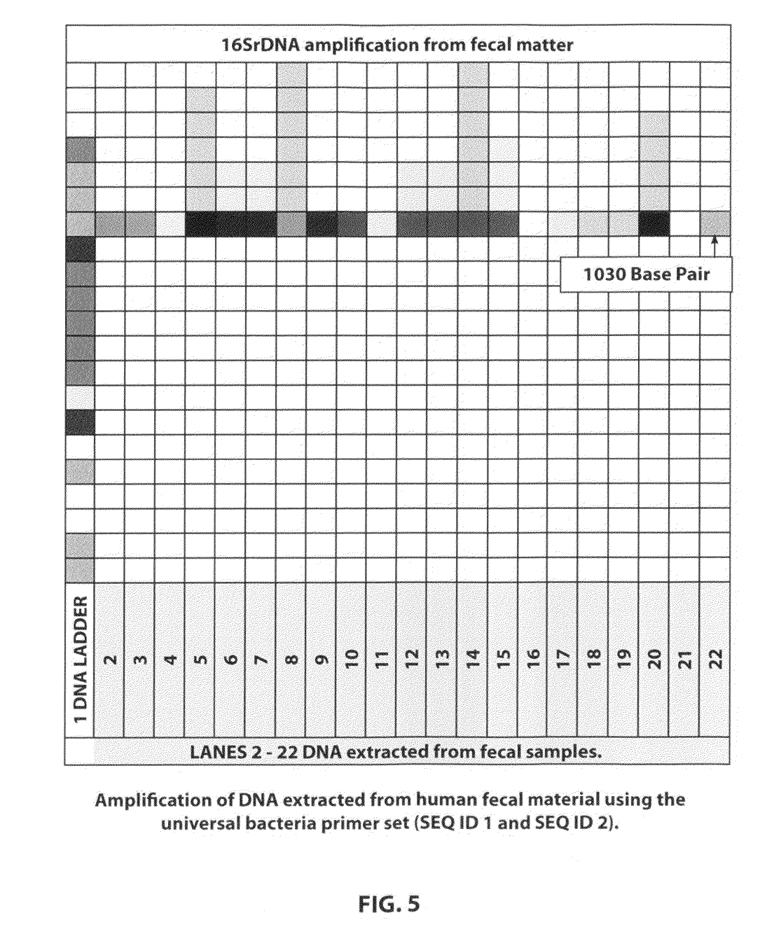

The bacterial universal primer pairs were used to amplify DNA extracted from 21 human fecal samples and the results are shown in FIG. 5. The PCRs were carried out according to methods detailed in Sambrook et al. (1989) at an annealing temperature of 55 degrees C. The results depicted in FIG. 5 are tabulated below in Table 5.

TABLE 5
Amplification of DNA extracted from human fecal material using
the universal bacteria primer set (SEQ ID 1 and SEQ ID 2).

| Lane # | Scoring |
|---|---|
| 1 | ++ |
| 2 | ++ |
| 3 | + |
| 4 | ++++ |
| 5 | +++ |
| 6 | +++ |
| 7 | ++ |
| 8 | ++++ |
| 9 | +++ |
| 10 | +/− |
| 11 | +++ |
| 12 | +++ |
| 13 | ++++ |
| 14 | +++ |
| 15 | − |
| 16 | + |
| 17 | ++ |
| 18 | ++ |
| 19 | ++++ |
| 20 | − |
| 21 | ++ |

The scorings for the agarose gel electrophoresis analysis is by quantitating the intensity of the PCR products in the stained gel using the naked eye. A definition of the scoring follows: − = no band; +/− = very faint band; + through ++++ = increasing intensity of the PCR products.

All of the compositions, processes and methods disclosed and claimed herein can be made and executed without undue experimentation in light of the present disclosure. While the compositions, processes and methods of this invention have been described in terms of preferred embodiments, it will be apparent to those of skill in the art that variations may be applied to the compositions, processes and methods and in the steps or in the sequence of steps of the methods described herein without departing from the concept, spirit, and scope of the invention. More specifically, it will be apparent that certain compositions, such as DNA sequences, primers, or probes, which are both chemically and physiologically related may be substituted for the compositions described herein while the same or similar results would be achieved. All such similar substitutes and modifications apparent to those skilled in the art are deemed to be within the spirit, scope, and concept of the invention.

What is claimed is:

1. A method for monitoring the microbiota of the human gastrointestinal tract, the method comprising the steps of;
identifying universal PCR primers to group microbial operational taxonomic units, and
applying the universal PCR primers to a sample of the gastrointestinal tract to produce PCR products between 500 bp-1500 bp in size.

2. The method of claim 1, wherein the universal PCR primers are specific to bacteria operational taxonomic units and comprises the sequence of any one of SEQ ID NO:1-SEQ ID NO:2 and SEQ ID NO. 54-SEQ ID NO. 55.

3. The method of claim 1, wherein the universal PCR primers are specific to fungi and yeast operational taxonomic units and comprises the sequence of any one of SEQ ID NO:82-SEQ ID NO:83 and SEQ ID NO:92-SEQ ID NO:93.

4. The method of claim 1, wherein the universal PCR primers are specific to parasitic protozoans and worms operational taxonomic units and comprises the sequence of any one of SEQ ID NO:92-SEQ ID NO:93.

5. The method of claim 2, 3, or 4 wherein qualitative or quantitative data is obtained and reported for specific microbial DNA sequences by analyzing DNA sequences of specific microbial operational taxonomic units using molecular-based methods, said molecular based methods comprising DNA hybridization, DNA arrays, DNA sequencing, PCR Arrays and multiplex PCR.

6. The method of claim 5, wherein oligonucleotides probes comprising sequences of any one of SEQ ID NO:1-SEQ ID NO:309 for the differentiation of microbes localized to the internal sequences of a specific operational taxonomic unit.

7. A process for monitoring microorganisms that are indigenous and/or pathogenic to an ecosystem, the process comprising: a) providing i) a method for simultaneous collection and inactivation of microbial growth in fecal material, ii) a method for extracting DNA from fecal material that is amendable to sensitive nucleic acid analysis, and iii) a method for concentrating target microbial nucleic acids; b) providing a method for the specific identification and quantification nucleic acid sequences specific to a microorganism at the genus or species level.

8. The process of claim 7, wherein the ecosystem of interest comprises the human gastrointestinal tract.

9. The process of claim 8, wherein fecal material is collected in medium containing 0.1%-50% formalin.

10. The process of claim 9, wherein the target nucleic acid is DNA.

11. A method for detecting a microbial species in a sample, said method comprising the steps of:
(a) lysing cells in said sample to release genomic DNA;
(b) contacting genomic DNA from step (a) with a primer pair comprising sequences of any one of SEQ ID NO:1-SEQ ID NO:309 for the differentiation of microbes localized to the internal sequences of a specific operational taxonomic unit;
(c) amplifying microbial DNA to produce an amplification product; and
(d) detecting said amplification product,
wherein the presence of said product is indicative of the presence of a microbial species in said sample and the absence of said product is indicative of the absence of a microbial species in said sample.

12. The method of claim 11 further comprising quantitating the level of a microbial species in the sample, said method comprising the steps of:
quantitating the level of said amplification product by comparison with at least one reference standard,
wherein the level of said amplification product is indicative of the level of said microbial species.


SEQUENCE LISTING (310 sequences)

SEQ ID NO: 1 - DNA, 19 bp, Artificial Sequence
  Conserved Bacteria Sequence
tcctacgggaggcagcagt

SEQ ID NO: 2 - DNA, 21 bp, Artificial Sequence
  Conserved Bacteria Sequence
taccttgttacgacttcaacc

SEQ ID NO: 3 - DNA, 18 bp, Artificial Sequence
  Acromabacter/Alcaligenes sp.
cagtttcacggggtatta

SEQ ID NO: 4 - DNA, 30 bp, Artificial Sequence
  Achromabacter/Alcaligenes sp.
cccaaataataccccgtgaaactgcccaaa

SEQ ID NO: 5 - DNA, 19 bp, Artificial Sequence
  Acinetobacter genus level primer
attctaccatcctctccca

SEQ ID NO: 6 - DNA, 31 bp, Artificial Sequence
  Acinetobacter genus level primer
ggaaggtgggagaggatggtagaataaggaa

SEQ ID NO: 7 - DNA, 20 bp, Artificial Sequence
  Aeromonas genus level primer
cgtctcaaggacacagcctc

SEQ ID NO: 8 - DNA, 32 bp, Artificial Sequence
  Genus level primer
cccaaagaggctgtgtccttgagacgcccaaa

SEQ ID NO: 9 - DNA, 21 bp, Artificial Sequence
  Genus level primer
tacaccaagaattccacctac

SEQ ID NO: 10 - DNA, 33 bp, Artificial Sequence
  Genus level primer
gggtttgtaggtggaattcttggtgtagggttt

SEQ ID NO: 11 - DNA, 20 bp, Artificial Sequence
  Genus level primer
catctgcctctccctcactc

SEQ ID NO: 12 - DNA, 32 bp, Artificial Sequence
  Genus level primer
gggtttgagtgagggagaggcagatggggttt

SEQ ID NO: 13 - DNA, 19 bp, Artificial Sequence
  Genus level primer
gtagggaggaaggtgtgag

SEQ ID NO: 14 - DNA, 21 bp, Artificial Sequence
  Genus level primer
taacaaaccacctgcatgcgc

SEQ ID NO: 15 - DNA, 33 bp, Artificial Sequence
  Genus level primer
tttggggcgcatgcaggtggtttgttagggttt

SEQ ID NO: 16 - DNA, 19 bp, Artificial Sequence
  Genus level primer
taccgtactctagctcagt

SEQ ID NO: 17 - DNA, 31 bp, Artificial Sequence
  Genus level primer
gggtttactgagctagagtacggtagggttt

SEQ ID NO: 18 - DNA, 19 bp, Artificial Sequence
  Genus level primer
gtcgcttccctttgtatac

SEQ ID NO: 19 - DNA, 31 bp, Artificial Sequence
  Genus level primer
cccaaagtatacaaagggaagcgaccccaaa

SEQ ID NO: 20 - DNA, 19 bp, Artificial Sequence
  Genus level primer
tcgcttcactttgtatctg

SEQ ID NO: 21 - DNA, 31 bp, Artificial Sequence
  Genus level primer
cccaaacagatacaaagtgaagcgacccaaa

SEQ ID NO: 22 - DNA, 20 bp, Artificial Sequence
  Genus level primer
aatcgttgatgatattagca

SEQ ID NO: 23 - DNA, 32 bp, Artificial Sequence
  Genus level primer
tttgggtgctaatatcatcaacgattgggttt

SEQ ID NO: 24 - DNA, 18 bp, Artificial Sequence
  Genus level primer
atggactttcacaccgga

SEQ ID NO: 25 - DNA, 30 bp, Artificial Sequence
  Genus level primer
ccttggtccggtgtgaaagtccatcctgtt

SEQ ID NO: 26 - DNA, 20 bp, Hafnia alvei
atttgaaactggtcagctag

SEQ ID NO: 27 - DNA, 32 bp, Hafnia alvei
ttccttctagctgaccagtttcaaatccttcc

SEQ ID NO: 28 - DNA, 18 bp, Artificial Sequence
  Genus level primer
ttcacgaagtcgggttgc

SEQ ID NO: 29 - DNA, 30 bp, Artificial Sequence
  Genus level primer
ccttccgcaacccgacttcgtgaaccttcc

SEQ ID NO: 30 - DNA, 17 bp, Artificial Sequence
  Genus level primer
agccggtccttattcat

SEQ ID NO: 31 - DNA, 29 bp, Artificial Sequence
  Genus level primer
ggttggatgaataaggaccggctggttgg

SEQ ID NO: 32 - DNA, 19 bp, Artificial Sequence
  Genus level primer
gagatccgccttcgccacc

SEQ ID NO: 33 - DNA, 31 bp, Artificial Sequence
  Genus level primer
gggtttggtggcgaaggcggatctcttggtt

SEQ ID NO: 34 - DNA, 20 bp, Artificial Sequence
  Genus level primer
agtatcgcatgagtccccaa

SEQ ID NO: 35 - DNA, 32 bp, Artificial Sequence
  Genus level primer
tttgggttggggactcatgcgatactggttgg

SEQ ID NO: 36 - DNA, 19 bp, Artificial Sequence
  Genus level primer
ggaaaccctctaacactta

SEQ ID NO: 37 - DNA, 31 bp, Artificial Sequence
  Genus level primer
cccaaataagtgttagagggtttcccccaaa

SEQ ID NO: 38 - DNA, 17 bp, Artificial Sequence
  Genus level primer
catgctccgctacttgt

SEQ ID NO: 39 - DNA, 29 bp, Artificial Sequence
  Genus level primer
ggttccacaagtagcggagcatgccggtt

SEQ ID NO: 40 - DNA, 19 bp, Enterococcus faecalis
actaacgtccttgttcttc

SEQ ID NO: 41 - DNA, 31 bp, Enterococcus faecalis
ggttgggaagaacaaggacgttagtttggtt

SEQ ID NO: 42 - DNA, 19 bp, Artificial Sequence
  Genus level primer
aataaaggccagttactac

SEQ ID NO: 43 - DNA, 31 bp, Artificial Sequence
  Genus level primer
ttggttgtagtaactggcctttattggttgg

SEQ ID NO: 44 - DNA, 19 bp, Artificial Sequence
  Genus level primer
agcagttactcttatcctt

SEQ ID NO: 45 - DNA, 31 bp, Artificial Sequence
  Genus level primer
ggttggaaggataagagtaactgctttggtt

SEQ ID NO: 46 - DNA, 20 bp, Artificial Sequence
  Genus level primer
caaatgcatgcccccggtta

SEQ ID NO: 47 - DNA, 32 bp, Artificial Sequence
  Genus level primer
ttggtttaaccgggggcatgcatttgggttgg

SEQ ID NO: 48 - DNA, 19 bp, Artificial Sequence
  Genus level primer
cgcatttcaccgcttcaca

SEQ ID NO: 49 - DNA, 31 bp, Artificial Sequence
  Genus level primer
tttgggtgtgaagcggtgaaatgcgtttggg

SEQ ID NO: 50 - DNA, 20 bp, Artificial Sequence
  Genus level primer
agaacttatagctccctaca

SEQ ID NO: 51 - DNA, 32 bp, Artificial Sequence
  Genus level primer
ggttggtgtagggagctataagttctttggtt

SEQ ID NO: 52 - DNA, 20 bp, Artificial Sequence
  Genus level primer
gtcagggtcagtccagacag

SEQ ID NO: 53 - DNA, 32 bp, Artificial Sequence
  Genus level primer
ccttccctgtctggactgaccctgacttcctt

SEQ ID NO: 54 - DNA, 20 bp, Artificial Sequence
  Genus level primer
tgaccgatagtgaaccagta

SEQ ID NO: 55 - DNA, 23 bp, Artificial Sequence
  Genus level primer
tttcgctaccttaggaccgttat

SEQ ID NO: 56 - DNA, 19 bp, Artificial Sequence
  Genus level primer
aatcacttcacctacgtgt

SEQ ID NO: 57 - DNA, 31 bp, Artificial Sequence
  Genus level primer
gggtttacacgtaggtgaagtgattgggttt

SEQ ID NO: 58 - DNA, 19 bp, Citrobacter freundii
acccaacaacacataagtg

SEQ ID NO: 59 - DNA, 31 bp, Citrobacter freundii
tttgggcacttatgtgttgttgggtgggttt

SEQ ID NO: 60 - DNA, 18 bp, Escherichia coli
acccaacaacacacagtg

SEQ ID NO: 61 - DNA, 30 bp, Escherichia coli
gggtttcactgtgtgttgttgggtgggttt

SEQ ID NO: 62 - DNA, 20 bp, Klebsiella pneumoniae
acgcagtcacacccgaaggt

SEQ ID NO: 63 - DNA, 32 bp, Klebsiella pneumoniae
gggtttaccttcgggtgtgactgcgtgggttt

SEQ ID NO: 64 - DNA, 20 bp, Klebsiella oxytoca
ttcacttaccatcagcgtgc

SEQ ID NO: 65 - DNA, 32 bp, Klebsiella oxytoca
gggaaagcacgctgatggtaagtgaagggaaa

SEQ ID NO: 66 - DNA, 21 bp, Staphylococcus aureus
gggcacctattttctatctag

SEQ ID NO: 67 - DNA, 33 bp, Staphylococcus aureus
cccaaactagatagaaaataggtgcccaaaccc

SEQ ID NO: 68 - DNA, 19 bp, Artificial Sequence
  Genus level primer
tgtgaaatcaacgactcga

SEQ ID NO: 69 - DNA, 31 bp, Artificial Sequence
  Genus level primer
tttccttcgagtcgttgatttcacacccttt

SEQ ID NO: 70 - DNA, 21 bp, Artificial Sequence
  Genus level primer
ccatcgcaattacaagtcgtg

SEQ ID NO: 71 - DNA, 33 bp, Artificial Sequence
  Genus level primer
tttgggcacgacttgtaattgcgatggtttggg

SEQ ID NO: 72 - DNA, 20 bp, Artificial Sequence
  Genus level primer
tacctgaacttcaccctgga

SEQ ID NO: 73 - DNA, 32 bp, Artificial Sequence
  Genus level primer
tttccgtccagggtgaagttcaggtatttccc

SEQ ID NO: 74 - DNA, 18 bp, Artificial Sequence
  Genus level primer
cacttctatgcgttccag

SEQ ID NO: 75 - DNA, 30 bp, Artificial Sequence
  Genus level primer
tttgggctggaacgcatagaagtgtttggg

SEQ ID NO: 76 - DNA, 18 bp, Artificial Sequence
  Genus level primer
cccggatttacctaagat

SEQ ID NO: 77 - DNA, 30 bp, Artificial Sequence
  Genus level primer
cccaaaatcttaggtaaatccgggaaaccc

SEQ ID NO: 78 - DNA, 18 bp, Proteus mirabilis
agggactttacctaccgc

SEQ ID NO: 79 - DNA, 30 bp, Proteus mirabilis
tttggggcggtaggtaaagtcccttttggg

SEQ ID NO: 80 - DNA, 20 bp, Proteus vulgaris
ggtcttcccggcttccgatt

SEQ ID NO: 81 - DNA, 32 bp, Proteus vulgaris
aaagggaatcggaagccgggaagaccaaaggg

SEQ ID NO: 82 - DNA, 19 bp, Artificial Sequence
  Genus level primer
aggatagaggcctaccatg

SEQ ID NO: 83 - DNA, 21 bp, Artificial Sequence
  Genus level primer
acttgcgcttactaggaattc

SEQ ID NO: 84 - DNA, 20 bp, Artificial Sequence
  Genus level primer
atgctaacagattcaagcgt

SEQ ID NO: 85 - DNA, 32 bp, Artificial Sequence
  Genus level primer
gtgtgtacgcttgaatctgttagcatggttgg

SEQ ID NO: 86 - DNA, 20 bp, Artificial Sequence
  Genus level primer
ggatttccaacggggccttt

SEQ ID NO: 87 - DNA, 32 bp, Artificial Sequence
  Genus level primer
aaagggaaaggccccgttggaaatccgggaaa

SEQ ID NO: 88 - DNA, 20 bp, Artificial Sequence
  Genus level primer
cgagacctaataggccccgt

SEQ ID NO: 89 - DNA, 32 bp, Artificial Sequence
  Genus level primer
aaagggacggggcctattaggtctcgaaaggg

SEQ ID NO: 90 - DNA, 19 bp, Artificial Sequence
  Species-specific primer
aatgaagccaacaaaaatg

SEQ ID NO: 91 - DNA, 31 bp, Artificial Sequence
  Genus level primer
gggtttcatttttgttggcttcattgggttt

SEQ ID NO: 92 - DNA, 21 bp, Artificial Sequence
  Genus level primer
gcaagtctggtgccagcagcc

SEQ ID NO: 93 - DNA, 20 bp, Artificial Sequence
  Genus level primer
ctaagggcatcacagacctg

SEQ ID NO: 94 - DNA, 18 bp, Blastocystis hominis
tcggtatcgtttatagct

SEQ ID NO: 95 - DNA, 30 bp, Blastocystis hominis
aaagggagctataaacgataccgagggaaa

SEQ ID NO: 96 - DNA, 23 bp, Blastocystis hominis
agcaacgcatatcacaatatacg

SEQ ID NO: 97 - DNA, 19 bp, Blastocystis hominis
gaagtatcagctatcacaa

SEQ ID NO: 98 - DNA, 19 bp, Blastocystis hominis
gaggattcctatagaatac

SEQ ID NO: 99 - DNA, 20 bp, Blastocystis hominis
agatacaatatatccaaaga

SEQ ID NO: 100 - DNA, 19 bp, Artificial Sequence
  Genus level primer
ccttatagtgtccggcccg

SEQ ID NO: 101 - DNA, 31 bp, Artificial Sequence
  Genus level primer
tttgggcgggccggacactataaggtttggg

SEQ ID NO: 102 - DNA, 20 bp, Taenia solium
cgcaggtgcagtggacaaac

SEQ ID NO: 103 - DNA, 32 bp, Taenia solium
tttcccgtttgtccactgcacctgcgtttccc

SEQ ID NO: 104 - DNA, 19 bp, Artificial Sequence
  Genus level primer
cgaacaggtgacgagcgac

SEQ ID NO: 105 - DNA, 31 bp, Artificial Sequence
  Genus level primer
gggtttgtcgctcgtcacctgttcgtttggg

SEQ ID NO: 106 - DNA, 20 bp, Artificial Sequence
  Genus level primer
catgctggagtattcaaggc

SEQ ID NO: 107 - DNA, 32 bp, Artificial Sequence
  Genus level primer
gggtttgccttgaatactccagcatgttggtt

SEQ ID NO: 108 - DNA, 17 bp, Artificial Sequence
  Genus level primer
gcccaagatgtctaagg

SEQ ID NO: 109 - DNA, 29 bp, Artificial Sequence
  Genus level primer
tttgggccttagacatcttgggcttggtt

SEQ ID NO: 110 - DNA, 22 bp, Artificial Sequence
  Genus level primer
aaacacgattgcctattccaac

SEQ ID NO: 111 - DNA, 20 bp, Artificial Sequence
  Genus level primer
tcccacaatttaaacatact

SEQ ID NO: 112 - DNA, 19 bp, Aeromonas hydrophila
gatcacctcgtcggagaac

SEQ ID NO: 113 - DNA, 20 bp, Aeromonas hydrophila
aacagccggactgccagcac

SEQ ID NO: 114 - DNA, 20 bp, Alcaligenes xylosoxydans
caactacgtcaagaaggacg

SEQ ID NO: 115 - DNA, 19 bp, Alcaligenes xylosoxydans
gccacgcggtagccgtcgg

SEQ ID NO: 116 - DNA, 20 bp, Candida kruisii
agctccgaatttttgacgag

SEQ ID NO: 117 - DNA, 20 bp, Ascaris lumbricoides
atgcattcgcagtcggacgt

SEQ ID NO: 118 - DNA, 19 bp, Candida albicans
gtaatggctaaatcagcat

SEQ ID NO: 119 - DNA, 20 bp, Candida albicans
agagtctgggcactcaataa

SEQ ID NO: 120 - DNA, 18 bp, Candida kruisii
gattgcctcagtaacggc

SEQ ID NO: 121 - DNA, 21 bp, Candida kruisii
tctcggtaggcagtatctccg

SEQ ID NO: 122 - DNA, 19 bp, Candida tropicalis
tgggtaaagttatgactgt

SEQ ID NO: 123 - DNA, 19 bp, Candida tropicalis
atccaagtcagcatacaat

SEQ ID NO: 124 - DNA, 22 bp, Citrobacter amalonaticus
ctrtaacgtcacggagttcgaa

SEQ ID NO: 125 - DNA, 21 bp, Citrobacter amalonaticus
cacgctgcggaatgttgttag

SEQ ID NO: 126 - DNA, 20 bp, Artificial Sequence
  Genus level primer
tgttctacatgtatgttccg

SEQ ID NO: 127 - DNA, 20 bp, Artificial Sequence
  Genus level primer
tgaacaacaacatcagggcg

SEQ ID NO: 128 - DNA, 19 bp, Dientamoeba fragilis
tcgtactgccggagaaggc

SEQ ID NO: 129 - DNA, 20 bp, Dientamoeba fragilis
gggtcaaaacatttcgctgt

SEQ ID NO: 130 - DNA, 18 bp, Escherichia coli
tgcgtttccgtgcgctct

SEQ ID NO: 131 - DNA, 20 bp, Escherichia coli
cacgctgaacatacagtttc

SEQ ID NO: 132 - DNA, 19 bp, Escherichia coli
tgaacgggaggtacgtgat

SEQ ID NO: 133 - DNA, 21 bp, Escherichia coli
cgataaatcgctcgatgaagc

SEQ ID NO: 134 - DNA, 20 bp, Escherichia coli
tatctgccatcatacccggc

SEQ ID NO: 135 - DNA, 20 bp, Escherichia coli
atctggccgccctggctacg

SEQ ID NO: 136 - DNA, 22 bp, Edwardsiella tarda
taagctggtagcgctgttcggc

SEQ ID NO: 137 - DNA, 21 bp, Edwardsiella tarda
cagtacttccagaatcttgag

SEQ ID NO: 138 - DNA, 20 bp, Artificial Sequence
  Genus level primer
ggtataggtggacaaagaac

SEQ ID NO: 139 - DNA, 20 bp, Artificial Sequence
  Genus level primer
aagtcgtcctcttgccaaag

SEQ ID NO: 140 - DNA, 21 bp, Artificial Sequence
  Genus level primer
atatgccaagagaattgtaga

SEQ ID NO: 141 - DNA, 20 bp, Artificial Sequence
  Genus level primer
tctcgaaacgatcatggaat

SEQ ID NO: 142 - DNA, 19 bp, Entamoeba histolytica
ccaggtaaatgtataggag

SEQ ID NO: 143 - DNA, 19 bp, Entamoeba histolytica
tcatgtttagattcagaag

SEQ ID NO: 144 - DNA, 21 bp, Enterobacter aerogenes
ccgataatggaaaaatacttc

SEQ ID NO: 145 - DNA, 19 bp, Enterobacter aerogenes
tcatccgtcacctcgtccg

SEQ ID NO: 146 - DNA, 20 bp, Enterobacter agglomerans
cgcgctctggtgccgttgac

SEQ ID NO: 147 - DNA, 21 bp, Enterobacter agglomerans
ccgaccacgttatcgccctct

SEQ ID NO: 148 - DNA, 19 bp, Artificial Sequence
  Genus level primer
cgtggttgcaggtggtggc

SEQ ID NO: 149 - DNA, 20 bp, Artificial Sequence
  Genus level primer
gacccgctacggacgccgca

SEQ ID NO: 150 - DNA, 19 bp, Artificial Sequence
  Genus level primer
cgtaggcgtttccgtggtt

SEQ ID NO: 151 - DNA, 19 bp, Artificial Sequence
  Genus level primer
tttgataccaccttcgtaa

SEQ ID NO: 152 - DNA, 20 bp, Enterobacter cloacae
tcaggggccagacaatcacc

SEQ ID NO: 153 - DNA, 20 bp, Enterobacter cloacae
tgccatgacgggcggcgatt

SEQ ID NO: 154 - DNA, 19 bp, Artificial Sequence
  Genus level primer
ctggtgatcaaccgcgaag

SEQ ID NO: 155 - DNA, 20 bp, Artificial Sequence
  Genus level primer
ggaagtgatcctctttaccg

SEQ ID NO: 156 - DNA, 20 bp, Enterobacter sakazakii
gtaggcgtttccgtcgtaaa

SEQ ID NO: 157 - DNA, 20 bp, Enterobacter sakazakii
aatggaaatgatcctctttg

SEQ ID NO: 158 - DNA, 18 bp, Artificial Sequence
  Genus level primer
tatcaaagttgcgctgcg

SEQ ID NO: 159 - DNA, 20 bp, Artificial Sequence
  Genus level primer
gcagaacgggtcactttagt

SEQ ID NO: 160 - DNA, 19 bp, Artificial Sequence
  Genus level primer
tggcgtctccgttgtaaac

SEQ ID NO: 161 - DNA, 18 bp, Artificial Sequence
  Genus level primer
aacgccctgatgccgcct

SEQ ID NO: 162 - DNA, 20 bp, Enterobius vermicularis
aaaggacccaatggcacaat

SEQ ID NO: 163 - DNA, 20 bp, Enterobius vermicularis
acagcagctaacaaataacg

SEQ ID NO: 164 - DNA, 18 bp, Escherichia coli
gaagatttcgggctcact

SEQ ID NO: 165 - DNA, 18 bp, Escherichia coli
aatcgcaccctgttcaac

SEQ ID NO: 166 - DNA, 18 bp, Escherichia fergusonii
atgcgttgccattaaccg

SEQ ID NO: 167 - DNA, 20 bp, Escherichia fergusonii
tcactgctgcgagggatgcg

SEQ ID NO: 168 - DNA, 20 bp, Escherichia hermannii
gggtgtatcggtggttaacg

SEQ ID NO: 169 - DNA, 20 bp, Escherichia hermannii
ggtagagaaatagaaaatgt

SEQ ID NO: 170 - DNA, 18 bp, Escherichia vulneris
gcagtgaccggcgatact

SEQ ID NO: 171 - DNA, 19 bp, Escherichia vulneris
cactggagcgcaacttcaa

SEQ ID NO: 172 - DNA, 21 bp, Giardia intestinalis
ggctccgcttccacccctctg

SEQ ID NO: 173 - DNA, 19 bp, Giardia intestinalis
atctcctccaggaagtaga

SEQ ID NO: 174 - DNA, 19 bp, Artificial Sequence
  Genus level primer
gcggcggacggctcaggac

SEQ ID NO: 175 - DNA, 18 bp, Artificial Sequence
  Genus level primer
tctggtggtacccctccg

SEQ ID NO: 176 - DNA, 20 bp, Helicobacter pylori
aatccattttagagcgcttg

SEQ ID NO: 177 - DNA, 20 bp, Helicobacter pylori
tgatattttttaacgacttc

SEQ ID NO: 178 - DNA, 20 bp, Artificial Sequence
  Genus level primer
aacaatgtccctgctacccg

SEQ ID NO: 179 - DNA, 19 bp, Artificial Sequence
  Genus level primer
cctggtacttatctggcag

SEQ ID NO: 180 - DNA, 19 bp, Klebsiella rhinoscleromatis
atatgacatcctggtgaaa

SEQ ID NO: 181 - DNA, 17 bp, Klebsiella rhinoscleromatis
agccgtcgttccactgc

SEQ ID NO: 182 - DNA, 20 bp, Kluyvera cryocrescens
atccacaaacagatttatgc

SEQ ID NO: 183 - DNA, 18 bp, Kluyvera cryocrescens
agatatttgggtggattg

SEQ ID NO: 184 - DNA, 18 bp, Kluyvera ascorbata
ctaatgtcgtcgagttcg

SEQ ID NO: 185 - DNA, 19 bp, Kluyvera ascorbata
agatattttcctggaagcc

SEQ ID NO: 186 - DNA, 17 bp, Artificial Sequence
  Genus level primer
gaattgctgatccgccg

SEQ ID NO: 187 - DNA, 18 bp, Artificial Sequence
  Genus level primer
ttcgggtgaataggcgtt

SEQ ID NO: 188 - DNA, 17 bp, Morganella morganii
caacccgctgtcagaaa

SEQ ID NO: 189 - DNA, 18 bp, Morganella morganii
aatttcatcggtaacaac

SEQ ID NO: 190 - DNA, 19 bp, Escherichia coli
gcaggcactaacgtctggc

SEQ ID NO: 191 - DNA, 18 bp, Escherichia coli
acgccgtgactttttcaa

SEQ ID NO: 192 - DNA, 20 bp, Necator americanus
tgtagcttgtggacagtact

SEQ ID NO: 193 - DNA, 21 bp, Necator americanus
ttgcaaatgacacatccacat

SEQ ID NO: 194 - DNA, 18 bp, Providencia stuartii
aaacttattgccacggtg

SEQ ID NO: 195 - DNA, 18 bp, Providencia stuartii
atggcgatttcaacacca

SEQ ID NO: 196 - DNA, 20 bp, Saccharomyces cerevisiae
aggactagaagccaaaagcc

SEQ ID NO: 197 - DNA, 18 bp, Saccharomyces cerevisiae
ttaaataagatcaaacgc

SEQ ID NO: 198 - DNA, 19 bp, Salmonella typhi
ggctgcggttaaagcaccg

SEQ ID NO: 199 - DNA, 19 bp, Salmonella typhi
cagaagccgcgtattgcag

SEQ ID NO: 200 - DNA, 20 bp, Serratia fonticola
gaccggtaccaccctgcgct

SEQ ID NO: 201 - DNA, 19 bp, Serratia fonticola
cgctacttcaacaccgata

SEQ ID NO: 202 - DNA, 19 bp, Serratia marcescens
tagtttattcctatcaaga

SEQ ID NO: 203 - DNA, 19 bp, Serratia marcescens
ggaagcagttgcagacaat

SEQ ID NO: 204 - DNA, 20 bp, Serratia odorifera
ccagaccgaattccagtacg

SEQ ID NO: 205 - DNA, 19 bp, Serratia odorifera
attttcgcatcgttcgggt

SEQ ID NO: 206 - DNA, 19 bp, Artificial Sequence
  Genus level primer
actgtccgagaagctggaa

SEQ ID NO: 207 - DNA, 20 bp, Artificial Sequence
  Genus level primer
ttctttgtccatataggcgt

SEQ ID NO: 208 - DNA, 21 bp, Artificial Sequence
  Genus level primer
cactctgctactgcgactaaa

SEQ ID NO: 209 - DNA, 20 bp, Artificial Sequence
  Genus level primer
cgtttgttgagcgagcgagg

SEQ ID NO: 210 - DNA, 19 bp, Shigella sonnei
gtgactcgagtagcagcat

SEQ ID NO: 211 - DNA, 21 bp, Shigella sonnei
gcacgcagtaccgcacctgac

SEQ ID NO: 212 - DNA, 19 bp, Shigella flexneri
aggtcgcgctcagacccac

SEQ ID NO: 213 - DNA, 19 bp, Shigella flexneri
aagttccaacacccaagca

SEQ ID NO: 214 - DNA, 19 bp, Shigella dysenteriae
tcgagtgattgcagaggcg

SEQ ID NO: 215 - DNA, 19 bp, Shigella dysenteriae
cgagcagaatttacaggcg

SEQ ID NO: 216 - DNA, 19 bp, Shigella boydii
ggccggtcagactgaagtt

SEQ ID NO: 217 - DNA, 20 bp, Shigella boydii
agcagaacttgcaggcacct

SEQ ID NO: 218 - DNA, 20 bp, Artificial Sequence
  Genus level primer
tgtagacttgtgaatggttt

SEQ ID NO: 219 - DNA, 18 bp, Artificial Sequence
  Genus level primer
aattagaaccaattatag

SEQ ID NO: 220 - DNA, 20 bp, Artificial Sequence
  Genus level primer
tctgaatttagtttagtggt

SEQ ID NO: 221 - DNA, 18 bp, Artificial Sequence
  Genus level primer
gtactaattagaaccaat

SEQ ID NO: 222 - DNA, 20 bp, Streptococcus pyogenes
gcatctacgggaaaaacata

SEQ ID NO: 223 - DNA, 20 bp, Streptococcus pyogenes
cataaagtcagcaatcttcc

SEQ ID NO: 224 - DNA, 18 bp, Streptococcus agalactiae
agtcgctccacgatctaa

SEQ ID NO: 225 - DNA, 20 bp, Streptococcus agalactiae
gttgctgagcgtgtcacaat

SEQ ID NO: 226 - DNA, 20 bp, Streptococcus dysgalactiae
tacgtctccttcgtgtacct

SEQ ID NO: 227 - DNA, 18 bp, Streptococcus dysgalactiae
gtgaataccaaggttacg

SEQ ID NO: 228 - DNA, 19 bp, Streptococcus bovis
gaatatcgtcgcggacatg

SEQ ID NO: 229 - DNA, 18 bp, Streptococcus bovis
caccttcatagtgatagt

SEQ ID NO: 230 - DNA, 19 bp, Strongyloides stercoralis
tccaaagtgacggatcatt

SEQ ID NO: 231 - DNA, 18 bp, Strongyloides stercoralis
tcaacaagttggtgaaca

SEQ ID NO: 232 - DNA, 18 bp, Artificial Sequence
  Genus level primer
aactctggtgctaataca

SEQ ID NO: 233 - DNA, 18 bp, Artificial Sequence
  Genus level primer
agactggccctctgctag

SEQ ID NO: 234 - DNA, 21 bp, Trichuris trichiura
catatcggtctacggttaagc

SEQ ID NO: 235 - DNA, 20 bp, Trichuris trichiura
gaacttatgtacggctcgat

SEQ ID NO: 236 - DNA, 20 bp, Vibrio alginolyticus
gaagcaatggagcgtgtggg

SEQ ID NO: 237 - DNA, 19 bp, Vibrio alginolyticus
atgttgctagcgcttcgcc

SEQ ID NO: 238 - DNA, 19 bp, Vibrio cholerae
atttgagctttcaactcag

SEQ ID NO: 239 - DNA, 19 bp, Vibrio cholerae
atttcttcggcttgtgagc

SEQ ID NO: 240 - DNA, 19 bp, Vibrio fluvialis
gagactcttgattaccacc

SEQ ID NO: 241 - DNA, 19 bp, Vibrio fluvialis
ctgcgttgattgcatccgc

SEQ ID NO: 242 - DNA, 21 bp, Vibrio parahaemolyticus
aacgaaaccaccgtgatggaa

SEQ ID NO: 243 - DNA, 18 bp, Vibrio parahaemolyticus
caactgcagcgcgaatgt

SEQ ID NO: 244 - DNA, 21 bp, Artificial Sequence
  Genus level primer
gcgagctttgcaggtacttca

SEQ ID NO: 245 - DNA, 19 bp, Artificial Sequence
  Genus level primer
accttagtaatggctctac

SEQ ID NO: 246 - DNA, 20 bp, Yersinia enterocolitica
agtgtgaataacagacagaa

SEQ ID NO: 247 - DNA, 20 bp, Yersinia enterocolitica
taagggtataaacatagcca

SEQ ID NO: 248 - DNA, 19 bp, Escherichia coli
ccgagagaatggccggacg

SEQ ID NO: 249 - DNA, 20 bp, Escherichia coli
agcacgtatctcccgttcta

SEQ ID NO: 250 - DNA, 19 bp, Escherichia coli
cgcactagggcgctttggt

SEQ ID NO: 251 - DNA, 21 bp, Escherichia coli
atcacagtaacgcgcagccat

SEQ ID NO: 252 - DNA, 18 bp, Escherichia coli
cactgtgtgttgttgggt

SEQ ID NO: 253 - DNA, 20 bp, Escherichia coli
ttgttgcttcagcaccgtag

SEQ ID NO: 254 - DNA, 18 bp, Shigella flexneri
cggcagtcagctaccggc

SEQ ID NO: 255 - DNA, 20 bp, Shigella flexneri
atcatccaacgcatcgctaa

SEQ ID NO: 256 - DNA, 20 bp, Shigella boydii
ccgcgatatagttggccctg

SEQ ID NO: 257 - DNA, 19 bp, Shigella boydii
aaacgtaccagttagcgaa

SEQ ID NO: 258 - DNA, 19 bp, Shigella dysenteriae
caccggtgatatagttggc

SEQ ID NO: 259 - DNA, 21 bp, Shigella dysenteriae
gtgggataacgtgccgaaagc

SEQ ID NO: 260 - DNA, 19 bp, Artificial Sequence
  Genus level primer
gtgaaaggctacggctcaa

SEQ ID NO: 261 - DNA, 20 bp, Artificial Sequence
  Genus level primer
taacccccgacacctagtac

SEQ ID NO: 262 - DNA, 19 bp, Clostridium acetobutylicum
ggaaatgcaggcttcaaag

SEQ ID NO: 263 - DNA, 19 bp, Clostridium difficile
tttagaacctacacatagt

SEQ ID NO: 264 - DNA, 20 bp, Clostridium difficile
ctataatggtaacacatgac

SEQ ID NO: 265 - DNA, 18 bp, Artificial Sequence
  Genus level primer
tgcgcgattggatatgcc

SEQ ID NO: 266 - DNA, 17 bp, Artificial Sequence
  Genus level primer
ctggacagttttaaatg

SEQ ID NO: 267 - DNA, 19 bp, Aeromonas hydrophila
atcctggaaatcctgcaac

SEQ ID NO: 268 - DNA, 20 bp, Aeromonas hydrophila
agatagaggctgtgattggt

SEQ ID NO: 269 - DNA, 18 bp, Artificial Sequence
  Genus level primer
agacaggtgctgcacggc

SEQ ID NO: 270 - DNA, 17 bp, Artificial Sequence
  Genus level primer
aatatgtcccagttcgg

SEQ ID NO: 271 - DNA, 19 bp, Campylobacter jejuni
ctcacagggatttgatctt

SEQ ID NO: 272 - DNA, 22 bp, Campylobacter jejuni
gaaattgttatgagaagtgcta

SEQ ID NO: 273 - DNA, 21 bp, Artificial Sequence
  Genus level primer
gagtttaatacgctcaatcat

SEQ ID NO: 274 - DNA, 19 bp, Artificial Sequence
  Genus level primer
tcgcttcactttgtatctg

SEQ ID NO: 275 - DNA, 19 bp, Salmonella typhimurium
ggtgtaacggtaagcagcc

SEQ ID NO: 276 - DNA, 18 bp, Salmonella typhimurium
ctcatttgtcgaacgcgc

SEQ ID NO: 277 - DNA, 20 bp, Artificial Sequence
  Genus level primer
aacgatggctaataccgcat

SEQ ID NO: 278 - DNA, 22 bp, Artificial Sequence
  Genus level primer
ttcaaatgctattccgaggttg

SEQ ID NO: 279 - DNA, 20 bp, Artificial Sequence
  Genus level primer
tgatttgcttaattgcacca

SEQ ID NO: 280 - DNA, 18 bp, Artificial Sequence
  Genus level primer
ttgctcaacaccaaaccc

SEQ ID NO: 281 - DNA, 19 bp, Artificial Sequence
  Genus level primer
ggtgaacggacacactgga

SEQ ID NO: 282 - DNA, 20 bp, Artificial Sequence
  Genus level primer
tgataggacataggctgatc

SEQ ID NO: 283 - DNA, 21 bp, Helicobacter pylori
ttcaaatacgctgtaatggat

SEQ ID NO: 284 - DNA, 20 bp, Helicobacter pylori
cacttctaacgctgatgata

SEQ ID NO: 285 - DNA, 19 bp, Artificial Sequence
  Genus level primer
aggtgctaataccggataa

SEQ ID NO: 286 - DNA, 18 bp, Artificial Sequence
  Genus level primer
actctcctcttctgcact

SEQ ID NO: 287 - DNA, 18 bp, Lactobacillus acidophilus
aggctggttgtcagatag

SEQ ID NO: 288 - DNA, 20 bp, Lactobacillus acidophilus
aatcgcctactttatgaaca

SEQ ID NO: 289 - DNA, 17 bp, Artificial Sequence
  Genus level primer
gggtcgcgtcctatcag

SEQ ID NO: 290 - DNA, 18 bp, Artificial Sequence
  Genus level primer
atggactttcacaccgga

SEQ ID NO: 291 - DNA, 20 bp, Bifidobacterium longum
agccttctgcgctttctgct

SEQ ID NO: 292 - DNA, 20 bp, Bifidobacterium longum
acatatcgaacgccagacca

SEQ ID NO: 293 - DNA, 18 bp, Bifidobacterium bifidum
ttctatcggcgtgggatg

SEQ ID NO: 294 - DNA, 18 bp, Bifidobacterium bifidum
cgagccgcctacgagccc

SEQ ID NO: 295 - DNA, 19 bp, Staphylococcus aureus
agccactcaggacaagcat

SEQ ID NO: 296 - DNA, 19 bp, Staphylococcus aureus
tgtgaatacatagcatatc

SEQ ID NO: 297 - DNA, 18 bp, Staphylococcus aureus
ttcaacctgaccaagggt

SEQ ID NO: 298 - DNA, 18 bp, Artificial Sequence
  Genus level primer
gtcgatcatgttgccgta

SEQ ID NO: 299 - DNA, 30 bp, Artificial Sequence
  Genus level primer
ggaaggtacggcaacatgatcgacggaagg

SEQ ID NO: 300 - DNA, 18 bp, Artificial Sequence
  Genus level primer
tatgatcgcggttccaca

SEQ ID NO: 301 - DNA, 30 bp, Artificial Sequence
  Genus level primer
ggaaggtgtggaaccgcgatcataaaggaa

SEQ ID NO: 302 - DNA, 19 bp, Artificial Sequence
  Genus level primer
tcacaaccgaaatagactg

SEQ ID NO: 303 - DNA, 22 bp, Artificial Sequence
  Genus level primer
gttaaaaagctcgtagttgaac

SEQ ID NO: 304 - DNA, 23 bp, Artificial Sequence
  Genus level primer
ctctcaatctgtcaatccttatt

SEQ ID NO: 305 - DNA, 22 bp, Artificial Sequence
  Genus level primer
ggttctattttgttggtttcta

SEQ ID NO: 306 - DNA, 23 bp, Artificial Sequence
  Genus level primer
ctctcaatctgtcaatccttatt

SEQ ID NO: 307 - DNA, 20 bp, Artificial Sequence
  Genus level primer
aactggaggaaggtggggat

SEQ ID NO: 308 - DNA, 18 bp, Artificial Sequence
  Genus level primer
aggaggtgatccaaccgc

SEQ ID NO: 309 - DNA, 21 bp, Artificial Sequence
  Genus level primer
gtcaataaaagaacaacaacc

SEQ ID NO: 310 - DNA, 19 bp, Artificial Sequence
  Genus level primer
tttccaacggggcctttcc